(19) United States
(12) Patent Application Publication
(10) Pub. No.: US 2007/0092546 A1
(43) Pub. Date: Apr. 26, 2007

(54) Thiamin based insect repellant

(75) Inventors: Lawrence S. Spiegel, East Sandwich, MA (US); Mary A. Spiegel, East Sandwich, MA (US)

Correspondence Address: LATIMER IP LAW, LLP, 13873 PARK CENTER ROAD, SUITE 122, HERNDON, VA, 20171, US

(73) Assignee: Violet Fire, LLC

(21) Appl. No.: 11/520,253

(22) Filed: Sep. 13, 2006

Related U.S. Application Data
(60) Provisional application No. 60/729,690, filed on Oct. 24, 2005.

(51) Int. Cl. A01N 43/78; A61K 8/29; A61K 8/27; A61K 8/25

(52) U.S. Cl. 424/405; 424/59; 514/276

(57) ABSTRACT

The present invention provides a topical application of thiamin to human skin or mammalian coats to provide effective insect repellency. Any commercially available thiamin compounds such as thiamin hydrochloride or thiamin mononitrate can be combined with a pharmaceutically or cosmetically acceptable carrier for preparing a composition for topical application. This composition and method of application can further be combined with sun-block or sunscreen agents to provide protection against insects as well as the harmful effects of UV radiation. This composition may also contain botanical oils or extracts to mask the thiamin odor and provide repellant synergy.

### PRIORITY

This application takes priority from U.S. Provisional Patent Application #60/729,690, titled “Thiamin Based Insect Repellant”, filed on Oct. 25, 2005.

### FIELD OF INVENTION

The current invention involves the use of Thiamin (Vitamin B1) for application to the skin of humans and mammals, the primary use of which is as an insect repellent. The thiamine can also be combined with other topical medicinal or other lotions, ointments, sprays, cremes, oils (including essential oils), and also with sunscreen or sun-blocking agents, to provide those topical substances with a means to repel insects.

### BACKGROUND OF THE INVENTION

Until recently the most effective and available insect repellant has been DEET (diethyltoluamide). It has been a major insect repellant component in commercial preparations for many years. The major deficiency with the use of DEET includes its strong toxicity not only for insects but also for mammals and specifically for humans. As a result of the toxicity issue, DEET concentrations are minimized in preparations of skin application. All repellants or other products that include repellants with DEET carry warnings against excessive application, especially for young children and people with sensitive skin.

There are a number of known preparations that utilize odor to repel insects. These include oils and extracts of citronella, catnip, lemon grass, germanium, cedar, rosemary, garlic, peppermint, thyme, cinnamon, pennyroyal and other odorous materials. All of these are relatively weak insect repellants and they are dissipated quickly because of their volatility, but act in synergy with thiamin for humans and mammals.

U.S. Pat. No. 4,049,828 explains the use of citral as an effective insecticide and insect repellant.

U.S. Pat. No. 4,127,672 explains a method to produce insect repellants having increased effectiveness, increased mucus tolerance and compatibility with plastic objects.

U.S. Pat. No. 5,614,558, U.S. Pat. No. 5,998,475 and U.S. Pat. No. 6,130,252 explain using aqueous compositions incorporating ethanolamine based compounds, either independently or in combination with ethanolamine based compounds being selected from simple compounds and conjugated compounds to produce a non-toxic, environmentally safe and easily employable insecticide and insect repellant.

U.S. Pat. No. 5,736,584 provides for an insect repelling composition that includes a mineral oil based carrier and an insect repelling amount of cactus extract made from the leaves and stem of the Prickly Pear cactus.

U.S. Pat. No. 5,711,953 claims an insect repellant made of biodegradable ingredients, primarily food products or foodstuff derivates. The insect repellant in this invention comprises two basic ingredients which are small amounts of garlic extract components and a hot pepper extract component in a surfactant and carrier solution.

European Patent Number 0 962 135 A1 relates to insect repellant composition containing ester, amide, urethane or their combination, as well as alcohol and a nonionic surfactant.

None of the prior art cited above explain the use of thiamine (vitamin B1) applied to skin for repelling insects.

Moreover, the thiamin based insect repellent can be combined with other sunscreen components for protection against UV radiation. As listed below, there are a number of citations available describing compositions that have effective protection against the harmful effects of ultraviolet radiation.

U.S. Pat. No. 5,547,659 discloses an improved topical composition having improved efficiency such as improved sunscreen effectiveness for protecting the skin from the harmful effect of ultraviolet radiation such as sunburn and sun-induced premature aging of the skin.

U.S. Pat. No. 5,552,135 describes improved sunscreen compositions and their methods of manufacture. The improved compositions include extracts of cereal plants and an optional sunscreen blocking agent. The compositions optionally may include dihydroxycinnamic derivatives.

U.S. Pat. No. 5,573,754 describes high SPF value sunscreen compositions having improved photo-protective properties by non-sunscreen mechanism. These compositions comprise allantoin, pantothenol, Vitamin E, its pharmaceutically-acceptable esters, or mixtures thereof, one or more sunscreen agents and carrier material.

U.S. Pat. No. 5,573,755 explains the use of hydroxyl salts which display ultraviolet absorption over at least a portion of wavelength range from 290 to 400 nanometers as sunscreen agents and sunscreen compositions comprising them together with a cosmetically acceptable vehicle.

### SUMMARY OF INVENTION

The present invention relates primarily to insect repelling compositions that optionally include combinations with sunscreen compositions having improved efficiency and efficacy for topical use while avoiding the harmful effects of excessive thiamin ingestion. The current invention includes a composition comprising:
(a) 0.10%-25% by weight of thiamin compound(s) like thiamin hydrochloride or thiamin mononitrate.
(b) optionally 0.1%-7% by weight of sun-block(s), sunscreen agent(s) or both,
(c) optionally, a safe and effective amount of sunless tanning agents, antimicrobial agents, de-pigmentation agents, anti-aging agents, antifungal agents, sunflower oil, vegetable oil, glycerin, coconut oil, stearic acid extracted from vegetable oil, beeswax, orange wax, tocopheryl acetate, buttermilk powder, sodium borate, xanthan gum, sucrose stearate, glucose, glucose oxidase, lactoperoxidase, rosemary extract, an essential oil containing fragrance and aloe or lanolin for skincare, and;
(c) a safe and effective amount of pharmaceutically and cosmetically acceptable topical carrier(s) which could be oil-based or water-based.

### DETAILED DESCRIPTION OF THE INVENTION

The current invention involves the use of Thiamin (Vitamin B1) as an effective insect repellant when supplied in appropriate amounts topically to mammalian skin which is especially effective in thwarting insects while remaining harmless for human beings.

It has been known for some time that ingestion of extremely high levels of thiamin results in insect repellency on the human skin. Ingested levels of doses of up to 100 mg/day for several weeks will repel mosquitoes and other biting insects from the skin. The concentration of 100 mg/day and that quantity consumed over a number of days can cause anaphylactic stock, allergic reactions and skin rashes. By contrast, the US FDA Recommended Daily Intake (RDI) of thiamin is up to 1.5 mg/day.

To alleviate the concern with ingestion, the development of a solution, cream, ointment, gel or other known carrier for application to the skin utilizing thiamin as a primary active component is desirable and the subject of the present invention. This invention is based on repellant preparations in which thiamin is used in suitable vehicles for direct skin or mammalian coat applications.

Thiamin is commercially available in two forms, as thiamin hydrochloride and thiamin mononitrate. Both have insect repellant properties and both can be formulated in vehicles for convenient skin application and are the subject of the present invention. Thiamin hydrochloride is soluble in water and glycerin and other polyhydric alcohols. Thiamin hydrochloride is not readily soluble in organic solvents. Thiamin mononitrate is not readily soluble with most commercially available solvents.

It is understood that the invention is not limited in its application to the details of the formulation and arrangement of the components as set forth in the following descriptions. The invention includes other embodiments and can be practiced or implemented in various ways. It should also be understood that the phraseology and terminology employed herein is for the purpose of description only and both to be regarded as limiting. The present invention may be better understood with reference to the examples and accompanying descriptions.

Following are several example formulations for preparing repellants of each form of commercially available thiamin.

### EXAMPLE 1

An aqueous thiamin-based lotion can be prepared by the following proportions by weight percentage:

| | |
|---|---|
| Thiamin Hydrochloride | 5.0% |
| | (acceptable in range of 0.10-15%) |
| Water | 94.3% |
| Carboxymethlycellulose | 0.5% |
| Methyl Paraben | 0.2% |
| | 100.0% |

For additional microbiological preservation and to potentiate the activity of the paraben preservation, a food-grade acid may be added to reduce the lotion pH to below 4.5. Non-irritating acids like phosphoric acid or d-gluconolactone, and organic acids like citric acid and lactic acid, may also be used.

The xanthan, cellulose gum or any other cold water swelling gum provides lotion viscosity to ease skin application without spillage. Methyl and propyl parabens are strong microbiological preservatives. Lactates, sorbates, benzoates, individually or in combination, can also be used in different combinations to achieve the same purpose.

The substitution of nisin (a natural antimicrobial agent used as a preservative in foods) or nisin and/or similar preservatives for parabens would make this example a natural formula. Other natural gums (i.e. guar or arabic) can be substituted or combined with xanthan gum. The quantity of gum can be adjusted to fulfill the desired properties of use (e.g. aerosol or pump dispersion or hand application).

### EXAMPLE 2

A thiamin-based lotion suitable for aerosol, pumping or pouring applications can be prepared by the following proportion of ingredients by weight:

| | |
|---|---|
| Thiamin Hydrochloride | 7.5% |
| | (in a range of 0.10-25%) |
| Water | 41.0% |
| Glycerin | 35.0% |
| Propylene Glycol | 15.0% |
| Xanthan Gum | 1.0% |
| Methyl Paraben | 0.5% |
| | 100.0% |

In this example, glycol and a preservative (e.g. methyl paraben) provide microbiological preservation. The presence of glycerin and glycol act as non-volatile solutions on the skin that allow for extended periods of protection.

### EXAMPLE 3

A cream type of thiamin-based lotion as an emulsion for suspension of thiamin mononitrate can be prepared by weight percentages as follows:

| | |
|---|---|
| Thiamin Mononitrate | 6.0% |
| | (in a range of 0.10-16%) |
| Vegetable Oil | 35.0% |
| Water | 58.3% |
| Xanthan Gum | 0.5% |
| Methyl Paraben | 0.2% |
| | 100.0% |

Again a food grade acid to pH below 4.5 can be implemented as required.

The oil-in-water emulsion is prepared by hydrating the xanthan gum in water, adding Thiamin and methyl paraben, and finally adding oil. High shear agitation or colloid milling can be used to create a stable emulsion. The Thiamin Mononitrate should be dispersed as a compound that is fine in particle size.

### EXAMPLE 4

A natural repellant can be prepared by dissolving thiamine hydrochloride in natural vegetable glycerin as follows:

| | |
|---|---|
| Thiamin hydrochloride | 2% |
| | (in the range of 0.10-15%) |
| Vegetable glycerin | 98% |
| | 100% |

Botanical oils or extracts may be added at 0.10-15.0% to provide repellant synergy and to mask the thiamin odor.

### EXAMPLE 5

A low viscosity pourable or pumpable repellant lotion can typically be prepared by weight percentages, as follows:

| | |
|---|---|
| Water | 74.7% |
| Thiamin Hydrochloride | 5.0 |
| | (in a range of 0.10-20%) |
| Repellant Aromatic Extracts | 5.0 |
| Hydrogenated Polydecene | 4.0 |
| Isocetyl Stearate | 3.0 |
| Glycerin | 2.0 |
| Cetearyl Alcohol | 2.0 |
| Sodium Behenoyl Lactate | 1.0 |
| Aloe Vera Oil | 1.0 |
| Glyceral Stearate | 1.0 |
| Tocopherol Acetate | 0.5 |
| Hydantoin | 0.3 |
| Methylparaben | 0.1 |
| Propylparaben | 0.1 |
| Non-volative | 100.0% |

### EXAMPLE 6

Thiamin may be incorporated into a typical high viscosity cream formulation for uniform skin application as a vanishing cream:

| | |
|---|---|
| Water (Deionized) | 65.8% |
| Stearic Acid | 16.0 |
| Thiamin Hydrochloride | 5.0 |
| | (in a range of 0.10-20%) |
| Sodium Behenoyl Lactate | 5.0 |
| Glycerin | 2.0 |
| Cetearyl Alcohol | 2.0 |
| Isopropyl Palmitate | 1.5 |
| Sodium Isostearoyl Lactate | 1.0 |
| Lanolin | 1.0 |
| DMDM Hydantoin | 0.2 |
| Methylparaben | 0.2 |
| Tocopherol Acetate | 0.2 |
| Propylparaben | 0.1 |
| | 100.0% |

Lanolin and aloe vera offer skin protection for extended periods of use. Aromatic repellant extracts may be added to mask the odor of thiamin and to provide an overall pleasant aroma. Aloe leaf extracts or extracted aloe oil and anhydrous lanolin or standard, moisture containing lanolin may be used for solubility requirements.

### EXAMPLE 7

Thiamin may be incorporated into a sunscreen gel as follows for an SPF-30 or higher formulation;

| | |
|---|---|
| Water (Deionized or distilled) | 76.0% |
| Ethylhexyl Methooxycinnamate | 17.0 |
| Thiamine Hydrochloride | 5.3 |
| | (in a range of 0.10-20%) |
| Carbomer | 1.0 |
| DMDM Hydantoin | 0.3 |
| Sodium Hydroxide | 0.4 |
| | 100.0% |

Aromatic botanical oils or extracts may be added to provide an overall pleasant aroma as well as a synergistic repellency.

### EXAMPLE 8

A cream type of thiamin-based lotion as an emulsion for suspension of thiamin mononitrate can be prepared by weight percentage as follows:

| | |
|---|---|
| Thiamin Mononitrate | 6.0 |
| | (in a range of 0.10-20%) |
| Vegetable Oil | 35.0 |
| Water | 58.2 |
| Xanthan Gum | 0.5 |
| Methyl Paraben | 0.2 |
| Propyl Paraben | 0.1 |
| | 100% |

Food grade acid to pH below 4.5.

The oil-in-water emulsion is prepared by hydrating the gum in water, adding thiamin and methyl paraben, and finally slowly adding oil. High shear agitation or colloid milling can be used to create a stable emulsion. The thiamine mononitrate should be dispersed as a compound that is fine in particle size.

Symbiotic repellency can be created by combining other repellants with certain aromatic botanical oils or extracts such as extracts of oils of citronella, catnip, garlic, peppermint, thyme, lemongrass, eucalyptus, cedar, pennyroyal, geranium, rosemary, lavender, and like other odorous materials. All of these are relatively weak insect repellants and they are dissipated quickly because of their volatility, but these botanical extracts may be added to thiamin preparations to mask the thiamin odor and to enhance repellant synergy. These botanical oils or extracts may all be added separately or in any combination to the working examples 1-8 provided above.

Symbiotic repellency can be created by combining other repellants within the type of thiamin preparations described in all examples for greater effectiveness. Thus for example, a quantity of 0.10%-15% by weight of citronella or catnip or other essential oil or extract is useful and sufficient. If added to the aqueous lotion of Example 1, for instance, they must be made soluble, potentially by the use of polysorbate or other equally useful surfactants.

The thiamin based compounds discussed in all examples (and shown in example 7) can be further combined with any one or more of the sunscreen agents including but not limited to seed-derived proteinaceous particulate material, extracts of cereal plants such as oats, corn, wheat barley, rye, rice and mixtures thereof and mixtures of octocrylene, ethylhexyl-p-methooxycinnamate, zinc oxide, and titanium dioxide. Many other sun-block and sunscreen agents are readily available for use with the current invention thereby providing a combined use of insect repellent and a sun-block and/or sunscreen agent.

Moreover a variety of auxiliary substances can be added to the type of thiamin preparations described in the working examples. These could include coloring materials and epidermal improvers such as lanolin, aloe vera and vitamin E. Use of any fragrances that attract the insects should be avoided. Repellant fragrances in combination with the other ingredients of the present compositions has repellance utility and masks the unpleasant thiamin odor.

What is claimed is:

1. A composition comprising thiamin and at least one of the following; water, or a pharmaceutically or cosmetically acceptable carrier or all, wherein said composition applied to a mammalian skin acts as a repellant toward insects, wherein said composition may be in any form including but not limited to a solid, liquid, aerosol, cream, lotion, ointment, micro-emulsion, solution or gel.

2. The composition of claim 1, wherein said thiamin is a commercially available form of thiamin compound(s) including but not limited to thiamin hydrochloride and thiamin mononitrate.

3. The composition of claim 1, wherein said composition at 25 C comprises 0.10% -25% by weight of said thiamin compound(s), 0.1%-20% by weight of topically active sun-block components or sunscreen active components or both and 55%-97.9% by weight of said carrier.

4. The composition of claim 1, wherein said composition includes agents comprising but not limited to sunless tanning agents, antimicrobial agents, de-pigmentation agents, anti-aging agents, antifungal agents, or any combination thereof, and wherein one or more of said agents may be topically active and topically applied.

5. The composition of claim 4, wherein said sun-blocking agents include but are not limited to inorganic sun-blocking agents such as zinc oxide, titanium dioxide, silica dioxide and magnesium dioxide and wherein said sun-blocking agents can also be nano-sized particles wherein said particle sizes are greater than or equal to 1 nano meter and said sizes comprise a particle size distribution.

6. The composition of claim 1, wherein said composition comprises at least one pigment that imparts a color other than white to said composition wherein said color either remains or substantially disappears during application to said skin.

7. The composition of claim 1, wherein said composition also contains amino acids, vitamin E or provitamins or both, lanolin, aloe vera, nucleoderivatives, vegetable extracts and water to form a white or other than white colored emulsion.

8. The composition of claim 7, wherein said composition includes but is not limited to cascara, frangula, chamomile, hyperic, calendula, elicriso, licorice, essential oils and any combination thereof

9. A method of protecting-mammalian skin from insects comprising topically applying to said skin a composition including an effective amount of thiamin and at least one pharmaceutically or cosmetically acceptable carrier or both, wherein said composition applied to said skin is acting as a repellant toward biting or stinging insects and wherein said composition may be in any form including but not limited to a solid, liquid, aerosol, cream, lotion, ointment, micro-emulsion, solution or gel.

10. The method of claim 9, wherein said composition includes a carrier that is water-based or oil-based or a combination thereof

11. The method of claim 9, wherein said thiamin is a commercially available form of thiamin compound(s) including but not limited to thiamin hydrochloride and thiamin mononitrate.

12. The method of claim 9, wherein said composition comprises 0.10%-25% by weight of said thiamin compound(s), 0.1%-20% by weight of topically active sun-blocking agents or sunscreen active agents or both and 55%-97.9% by weight of said carrier.

13. The method of claim 9, wherein said composition includes agents comprising but not limited to sunless tanning agents, antimicrobial agents, de-pigmentation agents, anti-aging agents, antimicrobial agents, or any combination thereof, and wherein one or more of said agents may be topically active and topically applied.

14. The method of claim 12, wherein said sun-blocking agents include but are not limited to inorganic sun-blocking agents such as zinc oxide, titanium dioxide, silica dioxide and magnesium dioxide.

15. The method of claim 14, wherein said sun-blocking agents are nano-sized particles wherein said particle sizes are greater than or equal to 1 nano meter and said sizes comprise a particle size distribution.

16. The method of claim 12, wherein said composition comprises at least one pigment that imparts a color other than white to said composition wherein said color either remains or substantially disappears during application to said skin.

17. The method of claim 9, wherein said composition also contains amino acids, vitamin E or provitamins or both, lanolin, aloe vera, nucleoderivatives, vegetable extracts and water to form a white or other than white colored emulsion.

18. The method of claim 9, wherein said composition includes but is not limited to cascara, frangula, chamomile, hyperic, calendula, elicriso, licorice, essential oils and any combination thereof

19. A method of protecting mammalian skin from insects and harmful ultra-violet radiation comprising topically applying to said skin a composition comprising 0.10%-25% by weight of said thiamin compound(s), 0.1%-20% by weight of topically active sun-blocking agents or sunscreen active agents or both and 55%-97.9% by weight of said carrier and wherein said composition includes agents comprising but not limited to sunblocking agents, sunless tanning agents, antimicrobial agents, de-pigmentation agents, anti-aging agents, antifungal agents, or any combination thereof, and wherein one or more of said agents may be topically active and topically applied.

20. A system for protecting mammalian skin from insects and optionally harmful ultra-violet radiation comprising topically applying to said skin a composition wherein adding botanical oil or extracts to thiamine preparations is providing a masking of thiamine odor and enhancing repellant synergy.

21. A system as in claim 20, wherein creating symbiotic repellency occurs by combining thiamine with extracts of citronella, catnip, garlic, peppermint, thyme, lemongrass, eucalyptus, germanium, pennyroyal, rosemary, cedar, lavender, and the like, while providing masking of said odor.
